(19) United States
(12) Patent Application Publication
(10) Pub. No.: US 2022/0334205 A1
(43) Pub. Date: Oct. 20, 2022

(54) Detection of Bio-Markers in Functional MRI Scans

(71) Applicant: Luis Andre Dutra e Silva, Brasilia (BR)

(72) Inventor: Luis Andre Dutra e Silva, Brasilia (BR)

(21) Appl. No.: 17/233,261

(22) Filed: Apr. 16, 2021

(51) Int. Cl. G01R 33/48; G01R 33/56; A61B 5/00

(52) U.S. Cl. CPC G01R 33/4806; G01R 33/56; A61B 5/7275; A61B 5/7246; A61B 5/7267

(57) ABSTRACT

An example system may include a processor and memory of a diagnostics server, wherein the processor is configured to perform one or more of provide a fMRI image of a brain taken over a period of time; generate a matrix representative of the fMRI image according to a matrix generation method obtained via a first training, the matrix having m rows and n columns, and having elements fᵢⱼ with 1≤i≤m and 1≤j≤n, wherein each element fᵢⱼ is indicative of blood oxygen levels of a j^{th} set of voxels of an i^{th} volumetric slice of the fMRI image; and use the matrix to obtain a set of first probabilities according to a deduction method obtained via a second training, each first probability being indicative of a correlation level between a corresponding mental condition in a list and the matrix.

### TECHNICAL FIELD

This application generally relates to analysis of functional MRI scans, and more particularly, to detection of bio-markers in functional MRI scans using artificial intelligence-based models.

### BACKGROUND OF THE INVENTION

Magnetic Resonance Imaging (MRI) scans of patient brains are widely used. Conventional MRI systems can detect a variety of conditions of the brain such as cysts, tumors, bleeding, swelling, developmental and structural abnormalities, infections, inflammatory conditions, or problems with the blood vessels. The MRI can determine if a shunt is working and detect damage to the brain caused by an injury or a stroke.

In some cases, a brain scan might be used to rule out other medical illnesses, such as a tumor, that could cause symptoms similar to a mental disorder, such as depression. However, as it comes to mental disorders, MRI scans of a brain cannot be used to diagnose a mental disorder, such as autism, anxiety, depression, schizophrenia, or bipolar disorder. Functional magnetic resonance imaging or functional MRI (fMRI) measures brain activity by detecting changes associated with blood oxygen consumption. This technique relies on the fact that cerebral blood oxygen consumption and neuronal activation are coupled. When an area of the brain is in use, blood oxygen consumption to that region also increases. However, conventional fMRI cannot be used for a reliable detection of mental disorders.

### BRIEF SUMMARY OF EMBODIMENTS OF THE INVENTION

Accordingly, a system and method for diagnosis of mental disorders based on bio-markers in functional MRI scans using artificial intelligence-based models are desired.

An aspect of some embodiments of the present invention relates to a system, comprising a processor of a diagnostics server node and a memory. The processor of a diagnostics server node is connected to an image processor of a functional MRI (fMRI) system and to a first computerized system and to a second computerized system. The memory is configured to store machine readable instructions that when executed by the processor, cause the processor to: (i) provide a fMRI image of a brain taken over a period of time, the fMRI image comprising m volumetric slices of the brain imaged at respective time steps in the period of time, each slice having same thickness and being subdivided into a plurality of voxels; (ii) generate a matrix representative of the fMRI image according to a matrix generation method obtained via a first training, the matrix having m rows and n columns, and having elements fᵢⱼ with 1≤i≤m and 1≤j≤n, wherein each element fᵢⱼ is indicative of blood oxygen levels of a j^{th} set of voxels of an i^{th} volumetric slice of the fMRI image; and (iii) use the matrix to obtain a set of first probabilities according to a deduction method obtained via a second training, each first probability being indicative of a correlation level between a corresponding mental condition in a list and the matrix.

In a variant, the instructions further cause the processor to: (iv) for each volumetric slice identify in the fMRI image a first set of N voxels having highest blood oxygen level and a second set of N voxels having lowest blood oxygen level, thereby obtain 2m sets of N voxels, where each voxel of the first set and of the second set corresponds to a respective unique region of the brain; (v) use the 2m sets of N voxels to identify 2 mN unique regions consisting of mN first unique regions in the brain with highest blood oxygen level and mN second unique regions in the brain with lowest blood oxygen level; (vi) compare the 2 mN unique regions to a predetermined atlas which correlates the predetermined mental conditions to groups of brain regions with highest blood oxygen level and with lowest blood oxygen level, in order to yield a set of second probabilities, each second probability being indicative of a similarity between a respective group of brain regions of the predetermined atlas corresponding to a respective mental condition in the list and the mN first unique regions with highest blood oxygen level and the mN second unique regions with lowest blood oxygen level in the fMRI image; and (vii) calculate a set of averages, each average being associated with a respective mental condition in the list, each average being an average of a respective first probability associated with the respective mental condition and a respective second probability associated with the respective mental condition.

The atlas may be a Talairach atlas.

In another variant, each average is a geometric average of the respective first probability associated with the respective mental condition and the respective second probability associated with the respective mental condition.

In yet another variant, the instructions further cause the processor to, prior to step (i): (a) train the first computerized system to generate the matrix representative of the fMRI image, by using a set of input fMRI images, according to the first training; and (b) train the second computerized system to use the matrix to obtain the set of first probabilities, according to the second training.

In some embodiments of the present invention, the first training comprises: (a1) providing the set of input fMRI images, each input fMRI image taken over a respective period of time, each input fMRI image comprising respective m first volumetric slices of the brain imaged at respective timesteps in the period of time, each first volumetric slice having same thickness and being subdivided into a plurality of first voxels, and each first voxel having a respective first location and a respective first blood oxygen level value; (a2) for each of the input fMRI images, instructing the first computerized system to generate a respective matrix having m rows and n columns according to predetermined first guidelines, the matrix having elements fᵢⱼ with 1≤i≤m and 1≤j≤n, wherein each element fᵢⱼ is indicative of blood oxygen levels of a j^{th} set of voxels of an i^{th} volumetric slice of the input fMRI image; (a3) using each matrix to infer an output representation of the respective input fMRI image, each output representation having m second volumetric slices which correspond to the respective m first volumetric slices, wherein each second volumetric slice is subdivided into a plurality of second voxels corresponding to the plurality of first voxels of the input fMRI image, each second voxel having a respective second location equal to the first location of the corresponding first voxel and a respective inferred blood oxygen level value; (a4) calculating an error value by comparing each inferred blood oxygen level value of each second voxel to the first blood oxygen level of the corresponding first voxel; (a5) if the error value is greater than a first predetermined threshold, reporting the error value to the first computerized system and instructing the first computerized system to repeat steps (a2) through (a4) to lower the error value by altering one or more parameters of the first guidelines, until the error value is lower than or equal to the first predetermined threshold; and (a6) if the error value is smaller than or equal to first predetermined threshold, ending training.

In a variant, the error value is a mean absolute percentage error (MAPE), where

$$MAPE = \frac{1}{z} {\sum }_{k = 1}^{z} \vert \frac{{A}_{k} - {F}_{k}}{{A}_{k}} \vert ,$$

where z is a total number of voxels in the set of input fMRI images, Fₖ is the inferred blood oxygen level value of a second voxel k, and Aₖ is the first blood oxygen level value of a first voxel corresponding to the second voxel k.

In some embodiments of the present invention, the second training comprises: (b1) after the first training is complete, receiving at least a subset of the set of matrices corresponding to input fMRI images of brains of patients that are known to have one or more of the predetermined mental conditions; (b2) instructing the second computerized system to use the matrices in the subset to predict whether each matrix corresponds to any of the one or more of the predetermined mental conditions, via second guidelines; (b3) calculating an accuracy value for the subset, by comparing predictions generated at (b2) to the known conditions corresponding to each matrix in the subset; (b4) if the accuracy value is lower than a second predetermined threshold, reporting the accuracy value to the second computerized system and instructing the second computerized system to repeat steps (b2) and (b3) to increase the accuracy value by altering one or more parameters of the second guidelines, until the accuracy value is greater than or equal to the second predetermined threshold; and (b5) if the accuracy value is greater than or equal to second predetermined threshold, ending training.

In a variant, the accuracy value is

$${F}_{1} = \text{?} ,$$

$$\text{?} \text{indicates text missing or illegible when filed}$$

tp is a number of correct predictions, fp is a number of false positive predictions, and fn is a number of false negative predictions.

Another aspect of some embodiments of the present invention relates to a method for detection of mental condition based on functional MRI scans, the method comprising: (i) receiving, by a diagnostics server, a fMRI image of a brain taken over a period of time, the fMRI image comprising m volumetric slices of the brain imaged at respective time steps in the period of time, each slice having same thickness and being subdivided into a plurality of voxels; (ii) generating, by the diagnostics server, a matrix representative of the fMRI image according to a matrix generation method obtained via a first training, the matrix having m rows and n columns, and having elements fᵢⱼ with 1≤i≤m and 1≤j≤n, wherein each element fᵢⱼ is indicative of blood oxygen levels of a j^{th} set of voxels of an i^{th} volumetric slice of the fMRI image; and (iii) using, by the diagnostics server, the matrix to obtain a set of first probabilities according to a deduction method obtained via a second training, each first probability being indicative of a correlation level between a corresponding mental condition in a list and the matrix.

In a variant the method further comprises: (iv) for each volumetric slice identifying in the fMRI image a first set of N voxels having highest blood oxygen level and a second set of N voxels having lowest blood oxygen level, thereby obtaining 2m sets of N voxels, where each voxel of the first set and of the second set corresponds to a respective unique region of the brain; (v) using the 2m sets of N voxels to identify 2 mN unique regions consisting of mN first unique regions in the brain with highest blood oxygen level and mN second unique regions in the brain with lowest blood oxygen level; (vi) comparing the 2 mN unique regions to a predetermined atlas which correlates the predetermined mental conditions to groups of brain regions with highest blood oxygen level and with lowest blood oxygen level, in order to yield a set of second probabilities, each second probability being indicative of a similarity between a respective group of brain regions of the predetermined atlas corresponding to a respective mental condition in the list and the mN first unique regions with highest blood oxygen level and the mN second unique regions with lowest blood oxygen level in the fMRI image; and (vii) calculating a set of averages, each average being associated with a respective mental condition in the list, each average being an average of a respective first probability associated with the respective mental condition and a respective second probability associated with the respective mental condition.

In a variant, the method further comprises, prior to step (i): (a) training a first computerized system to generate the matrix representative of the fMRI image, by using a set of input fMRI images, according to the first training; and (b) training a second computerized system to use the matrix to obtain the set of first probabilities, according to the second training.

In a variant, the first training comprises: (a1) providing the set of input fMRI images, each input fMRI image taken over a respective period of time, each input fMRI image comprising respective m first volumetric slices of the brain imaged at respective timesteps in the period of time, each first volumetric slice having same thickness and being subdivided into a plurality of first voxels, and each first voxel having a respective first location and a respective first blood oxygen level value; (a2) for each of the input fMRI images, instruct the first computerized system to generating a respective matrix having m rows and n columns according to predetermined first guidelines, the matrix having elements fᵢⱼ with 1≤i≤m and 1≤j≤n, wherein each element fᵢⱼ is indicative of blood oxygen levels of a j^{th} set of voxels of an i^{th} volumetric slice of the input fMRI image; (a3) using each matrix to infer an output representation of the respective input fMRI image, each output representation having m second volumetric slices which correspond to the respective m first volumetric slices, wherein each second volumetric slice is subdivided into a plurality of second voxels corresponding to the plurality of first voxels of the input fMRI image, each second voxel having a respective second location equal to the first location of the corresponding first voxel and a respective inferred blood oxygen level value; (a4) calculating an error value by comparing each inferred blood oxygen level value of each second voxel to the first blood oxygen level of the corresponding first voxel; (a5) if the error value is greater than a first predetermined threshold, reporting the error value to the first computerized system and instructing the first computerized system to repeat steps (a2) through (a4) to lower the error value by altering one or more parameters of the first guidelines, until the error value is lower than or equal to the first predetermined threshold; and (a6) if the error value is smaller than or equal to first predetermined threshold, ending training.

In a variant, the second training comprises: (b1) after the first training is complete, receiving at least a subset of the set of matrices corresponding to input fMRI images of brains of patients that are known to have one or more of the predetermined mental conditions; (b2) instructing the second computerized system to use the matrices in the subset to predict whether each matrix corresponds to any of the one or more of the predetermined mental conditions, via second guidelines; (b3) calculating an accuracy value for the subset, by comparing predictions generated at (b2) to the known conditions corresponding to each matrix in the subset; (b4) if the accuracy value is lower than a second predetermined threshold, reporting computerized system to repeat steps (b2) and (b3) to increase the accuracy value by altering one or more parameters of the second guidelines, until the accuracy value is greater than or equal to the second predetermined threshold; and (b5) if the accuracy value is greater than or equal to second predetermined threshold, ending training.

Yet another aspect of some embodiments of the present invention relates to a non-transitory computer readable medium comprising instructions, that when read by a processor, cause the processor to perform: (i) providing a fMRI image of a brain taken over a period of time, the fMRI image comprising m volumetric slices of the brain imaged at respective time steps in the period of time, each slice having same thickness and being subdivided into a plurality of voxels; (ii) generating a matrix representative of the fMRI image according to a matrix generation method obtained via a first training, the matrix having m rows and n columns, and having elements fᵢⱼ with 1≤i≤m and 1≤j≤n, wherein each element fᵢⱼ is indicative of blood oxygen levels of a j^{th} set of voxels of an i^{th} volumetric slice of the fMRI image; and (iii) using the matrix to obtain a set of first probabilities according to a deduction method obtained via a second training, each first probability being indicative of a correlation level between a corresponding mental condition in a list and the matrix. \

In a variant, the non-transitory computer readable medium further comprises instructions, that when read by the processor, cause the processor to: (iv) for each volumetric slice identify in the fMRI image a first set of N voxels having highest blood oxygen level and a second set of N voxels having lowest blood oxygen level, thereby obtain 2m sets of N voxels, where each voxel of the first set and of the second set corresponds to a respective unique region of the brain; (v) use the 2m sets of N voxels to identify 2 mN unique regions consisting of mN first unique regions in the brain with highest blood oxygen level and mN second unique regions in the brain with lowest blood oxygen level; (vi) compare the 2 mN unique regions to a predetermined atlas which correlates the predetermined mental conditions to groups of brain regions with highest blood oxygen level and with lowest blood oxygen level, in order to yield a set of second probabilities, each second probability being indicative of a similarity between a respective group of brain regions of the predetermined atlas corresponding to a respective mental condition in the list and the mN first unique regions with highest blood oxygen level and the mN second unique regions with lowest blood oxygen level in the fMRI image; and (vii) calculate a set of averages, each average being associated with a respective mental condition in the list, each average being an average of a respective first probability associated with the respective mental condition and a respective second probability associated with the respective mental condition.

In some embodiments of the present invention, the non-transitory computer readable medium further comprises instructions, that when read by the processor, cause the processor to: (a) train a first model to generate the matrix representative of the fMRI image, by using a set of input fMRI images, according to the first training; and (b) train a second model to use the matrix to obtain the set of first probabilities, according to the second training.

In a variant, training of the first model comprises: (a1) providing the set of input fMRI images, each input fMRI image taken over a respective period of time, each input fMRI image comprising respective m first volumetric slices of the brain imaged at respective timesteps in the period of time, each first volumetric slice having same thickness and being subdivided into a plurality of first voxels, and each first voxel having a respective first location and a respective first blood oxygen level value; (a2) for each of the input fMRI images, instruct the first computerized system to generating a respective matrix having m rows and n columns according to predetermined first guidelines, the matrix having elements fᵢⱼ with 1≤i≤m and 1≤j≤n, wherein each element fᵢⱼ is indicative of blood oxygen levels of a j^{th} set of voxels of an i^{th} volumetric slice of the input fMRI image; (a3) using each matrix to infer an output representation of the respective input fMRI image, each output representation having m second volumetric slices which correspond to the respective m first volumetric slices, wherein each second volumetric slice is subdivided into a plurality of second voxels corresponding to the plurality of first voxels of the input fMRI image, each second voxel having a respective second location equal to the first location of the corresponding first voxel and a respective inferred blood oxygen level value; (a4) calculating an error value by comparing each inferred blood oxygen level value of each second voxel to the first blood oxygen level of the corresponding first voxel; (a5) if the error value is greater than a first predetermined threshold, reporting the error value to the first computerized system and instructing the first computerized system to repeat steps (a2) through (a4) to lower the error value by altering one or more parameters of the first guidelines, until the error value is lower than or equal to the first predetermined threshold; and (a6) if the error value is smaller than or equal to first predetermined threshold, ending the training of the first model.

In a variant, the non-transitory computer readable medium further comprises instructions, that when read by the processor, cause the processor to calculate the error value as a mean absolute percentage error (MAPE), where

$$MAPE = \frac{1}{z} {\sum }_{k = 1}^{z} \vert \frac{{A}_{k} - {F}_{k}}{{A}_{k}} \vert ,$$

where z is a total number of voxels in the set of input fMRI images, Fₖ is the inferred blood oxygen level value of a second voxel k, and Aₖ is the first blood oxygen level value of a first voxel corresponding to the second voxel k.

In a variant, training of the second model comprises: (b1) after the training of the first model is complete, receiving at least a subset of the set of matrices corresponding to input fMRI images of brains of patients that are known to have one or more of the predetermined mental conditions; (b2) instructing the second model to use the matrices in the subset to predict whether each matrix corresponds to any of the one or more of the predetermined mental conditions, via second guidelines; (b3) calculating an accuracy value for the subset, by comparing predictions generated at (b2) to the known conditions corresponding to each matrix in the subset; (b4) if the accuracy value is lower than a second predetermined threshold, repeating steps (b2) and (b3) to increase the accuracy value by altering one or more parameters of the second guidelines, until the accuracy value is greater than or equal to the second predetermined threshold; and (b5) if the accuracy value is greater than or equal to second predetermined threshold, ending the training of the second model, wherein the accuracy value is

$${F}_{1} = \text{?} ,$$

$$\text{?} \text{indicates text missing or illegible when filed}$$

tp is a number of correct predictions, fp is a number of false positive predictions, and fn is a number of false negative predictions.

### BRIEF DESCRIPTION OF DRAWINGS

FIG. 1A illustrates a network diagram of a system including an AI module and a model database, according to example embodiments.

FIG. 1B illustrates a network diagram of a system including detailed features of a diagnostics server node, according to example embodiments.

FIG. 2 illustrates how fMRI system uses scanned layers, according to example embodiments.

FIG. 3 illustrates fMRI image processing for detection of mental conditions, according to example embodiments.

FIG. 4 illustrates a diagram of application of feature/step matrixes for detection of mental conditions, according to example embodiments.

FIG. 5A illustrates a flow diagram of a method, according to example embodiments.

FIG. 5B illustrates a further flow diagram of a method, according to example embodiments.

FIG. 5C illustrates a flow diagram of an example model training method, according to example embodiments.

FIG. 6 illustrates an example server system that supports one or more of the example embodiments.

### DETAILED DESCRIPTION OF THE EMBODIMENTS OF THE INVENTION

It will be readily understood that the instant components, as generally described and illustrated in the figures herein, may be arranged and designed in a wide variety of different configurations. Thus, the following detailed description of the embodiments of at least one of a method, apparatus, non-transitory computer readable medium and system, as represented in the attached figures, is not intended to limit the scope of the application as claimed but is merely representative of selected embodiments.

The instant features, structures, or characteristics as described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

In addition, while the term “message” may have been used in the description of embodiments, the application may be applied to many types of network data, such as, packet, frame, datagram, etc. The term “message” also includes packet, frame, datagram, and any equivalents thereof. Furthermore, while certain types of messages and signaling may be depicted in exemplary embodiments they are not limited to a certain type of message, and the application is not limited to a certain type of signaling.

Example embodiments provide methods, systems, components, non-transitory computer readable media, devices, and/or networks, which provide for diagnosis of mental disorders based on bio-markers in fMRI scans using artificial intelligence-based models.

The exemplary embodiment may use Deep Learning models for detection of bio-markers through the analysis of fMRI scans, demographic and psychometric data. In one embodiment an artificial intelligence (AI) machine learning systems may be employed for detection of biomarkers associated with mental conditions in fMRI scans of a brain.

Machine learning relies on vast quantities of historical data (or training data) to build predictive models for accurate prediction on new diagnosis-related data. Machine learning software may sift through millions of records to unearth non-intuitive patterns. In the example embodiment, a diagnostics platform may build and deploy a machine learning model for predictive monitoring and detection of mental conditions based on fMRI data. The diagnostics platform may be a cloud platform, a server, a web server, a personal computer, a user device attached to the fMRI system, and the like. A neural network or blockchain may be used to improve both a training process of the machine learning model and a predictive process based on a trained machine learning models. For example, rather than requiring a data scientist or a doctor or other user to collect the data, historical data may be stored on neural network or on the blockchain. This can significantly reduce the collection time needed by the diagnostics platform when performing predictive model training.

According to one embodiment, a U-Net 4D model may be used. This model works with sparse tensors which means that only coordinates and values of each voxel of the fMRI slice that are different from zero are used as an input to the neural network. Thus, the neural network can receive, as input, files of different sizes, without the need to do previous resampling. The only pre-processing may be the normalization of voxels to zero mean and standard deviation one.

Since the size of each file can be different from one iteration to another, the training must be done with only one sample per iteration. This makes the time to train each epoch to be about 48 hours. After almost 30 days of training, the network is able to represent any brain with a “fingerprint” matrix from 4D nifti file with only 9.6% of Mean Absolute Percentage Error (MAPE) in test data. Once a particular experiment is chosen a feature extraction may be performed. For every file in nifti 4D format, a forward pass to the U-Net is performed and the result may be stored as a numpy array for the next stage. This stage may take around 2 hours per unique diagnosis of a bio-marker.

Then, fMRI scans for a given patient may be classified. To classify each fMRI scan of a given patient as positive or negative according to the chosen biomarker, a Siamese LSTM network may be trained with the output of the U-Net from previous stage. In this stage, the extracted scan features and demographic or psychometric data may be used in the final model.

Accordingly, the exemplary embodiments provide for a specific solution to a problem in the arts/field of fMRI-based diagnostics. According to the exemplary embodiments, a method, system and a computer readable medium for detection of bio-markers in fMRI scans using artificial intelligence-based models are provided.

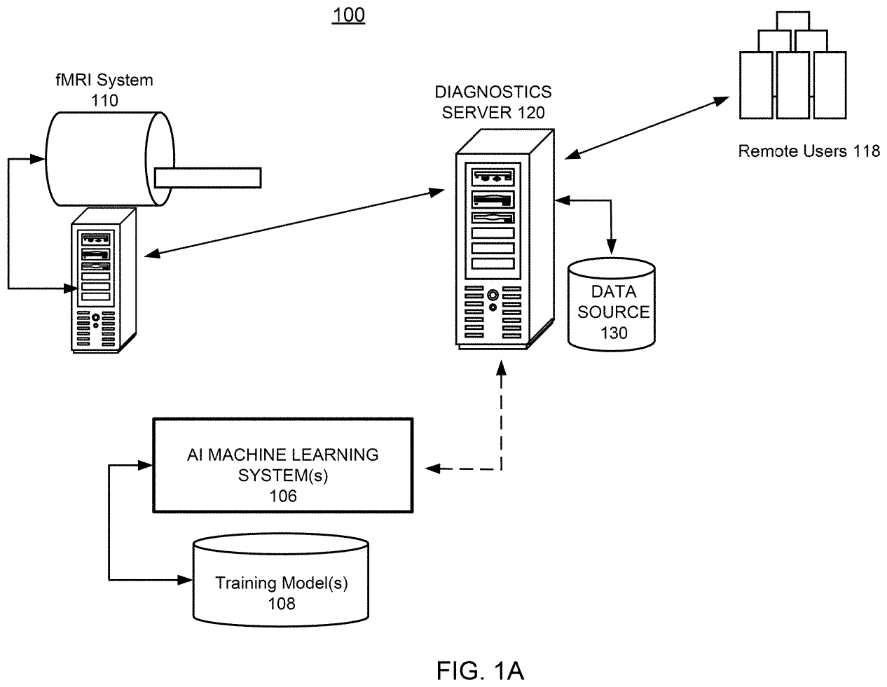

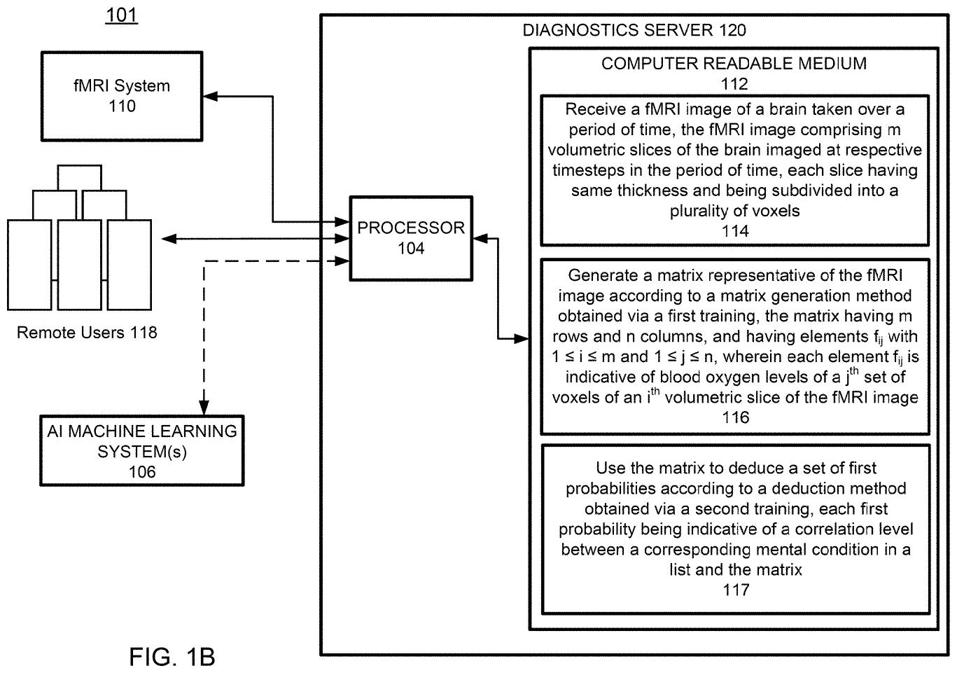

FIG. 1A illustrates a network diagram 100 of a system including an AI module and a model database, according to example embodiments.

Referring to FIG. 1A, a diagnostics server 120 may be to a fMRI system 110 over a network. The diagnostics server 120 may be connected to remote users (such as doctors) 118 over a network. In one embodiment, the diagnostics server 120 may be connected to AI machine learning systems 106. The diagnostics server 120 may provide training data from a data source 130 to train models 108 of the AI machine learning systems 106.

As discussed above, fMRI scans acquired from the fMRI system 110 for a given patient may be classified. To classify each fMRI scan of a given patient as positive or negative according to the chosen biomarker, a Siamese LSTM network residing on the AI machine learning systems 106 may be trained with the output of the data source 130 (e.g., U-Net). In this stage, the extracted scan features and demographic or psychometric data may be used in the final model 108 of the AI machine learning systems 106.

According to the exemplary embodiments, the diagnostics server 120 may execute the following algorithm:

1. Let F be the set of fMRI scans of the OpenNeuro database;
2. For each x in F, perform f(x) = x using a 4D Sparse U-Net;
3. Calculate Mean Absolute Percentage Error (MAPE);
4. If MAPE <10%, then continue, else go to step 2;
5. Let M be the representation model of individual brains;
6. Let G be the subset of brain representations of a particular study;
7. For each y in G, classified as c, perform g(y) = c′ using a LSTM network;
8. Calculate Accuracy;
9. If Accuracy >90%, then continue, else, go to step 7;
10. Let L be the classification model of a particular study
11. For each class c, obtain the centroid of c, calculating the average matrix;
12. Let T be the set of centroids of all diagnosis;
13. For each x, z in F, perform h(x, z) = 1 if i(x) = i(z), where i is an individual, else h(x, z) = 0;
14. Calculate Accuracy;
15. If Accuracy >99%, then continue, else go to step 13;
16. Let r be the fMRI scan performed by an individual;
17. Perform g(r) and obtain class c′;
18. For each t in T, Perform h(r, t) = v, where t is the centroid of class c′;
19. Get the maximum value of v and set the result as class c″;

FIG. 1B illustrates a network diagram 101 of a system including detailed features of an ad processing server node, according to example embodiments.

Referring to FIG. 1B, the example network 101 includes the diagnostics server 120 connected to the fMRI system 110 over a network. The diagnostics server 120 may be connected to remote users (such as doctors) 118 over a network. In one embodiment, the diagnostics server 120 may be connected to AI machine learning systems 106. The diagnostics server 120 may provide training data to train models of the AI machine learning systems 106.

While this example describes in detail only one diagnostics server 120, multiple such nodes may be connected to the fMRI system 110. It should be understood that the diagnostics server 120 may include additional components and that some of the components described herein may be removed and/or modified without departing from a scope of the diagnostics server 120 disclosed herein. The diagnostics server 120 may be a computing device or a server computer, or the like, and may include a processor 104, which may be a semiconductor-based microprocessor, a central processing unit (CPU), an application specific integrated circuit (ASIC), a field-programmable gate array (FPGA), and/or another hardware device. Although a single processor 104 is depicted, it should be understood that the diagnostics server 120 may include multiple processors, multiple cores, or the like, without departing from the scope of the diagnostics server 120 system.

The diagnostics server 120 may also include a non-transitory computer readable medium 112 that may have stored thereon machine-readable instructions executable by the processor 104. Examples of the machine-readable instructions are shown as 114-117 and are further discussed below. Examples of the non-transitory computer readable medium 112 may include an electronic, magnetic, optical, or other physical storage device that contains or stores executable instructions. For example, the non-transitory computer readable medium 112 may be a Random Access memory (RAM), an Electrically Erasable Programmable Read-Only Memory (EEPROM), a hard disk, an optical disc, or other type of storage device.

The processor 104 may fetch, decode, and execute the machine-readable instructions 114 to receive a fMRI image of a brain taken over a period of time, the fMRI image comprising m volumetric slices of the brain imaged at respective timesteps in the period of time, each slice having same thickness and being subdivided into a plurality of voxels. The processor 104 may fetch, decode, and execute the machine-readable instructions 116 to generate a matrix representative of the fMRI image according to a matrix generation method obtained via a first training, the matrix having m rows and n columns, and having elements fᵢⱼ with 1≤i≤m and 1≤j≤n, wherein each element fᵢⱼ is indicative of blood oxygen levels of a j^{th} set of voxels of an i^{th} volumetric slice of the fMRI image. Note that the fᵢⱼ is based on a concept of sparse convolutions in 4D volumes. The second AI may receive a point cloud formed by positive voxel values and may transform that volume into a lower dimensional representation using the frozen model of the first AI system.

The processor 104 may fetch, decode, and execute the machine-readable instructions 117 to use the matrix to obtain a set of first probabilities according to a deduction method obtained via a second training, each first probability being indicative of a correlation level between a corresponding mental condition in the list and the matrix.

According to the exemplary embodiments, the diagnostics server 120 may perform comparison of the matrix representative of the fMRI image with a plurality of matrices obtained in second training each of these matrices being representative of a respective condition. Therefore, the probability of the patient having schizophrenia, for example, is indicative of how closely the matrix generated by instructions 116 matches a matrix representative of schizophrenia that was obtained during the second training that is discussed in more details below.

Note that the probabilities for each unknown mental condition are calculated by taking all input matrices and treating them as multiple time series of m steps and n features. The second AI model learns to associate each matrix to the ground truth given by a specific study. It is a model that uses recurrent layers to transform a sequence of features into a unique feature that is the probability.

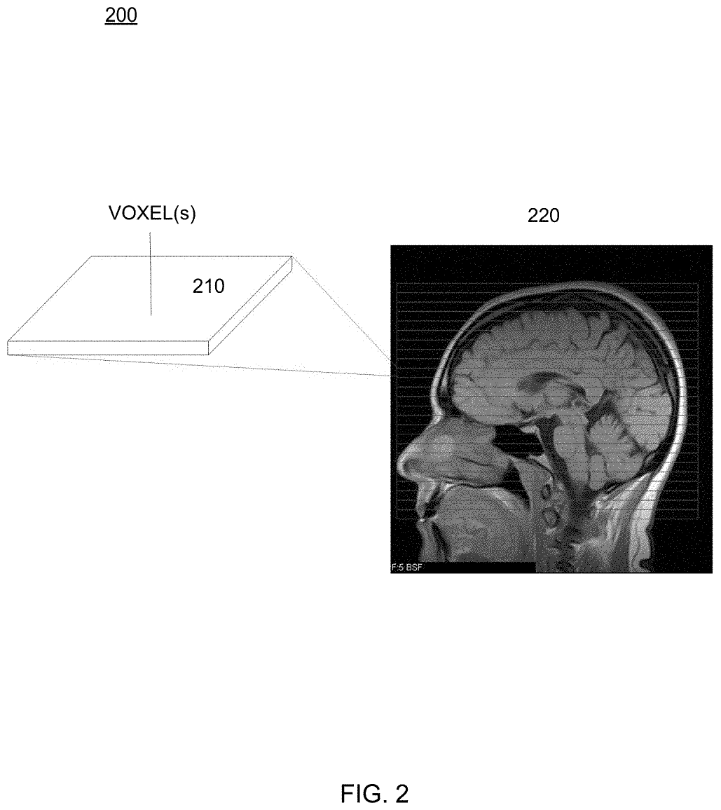

FIG. 2 illustrates how fMRI system uses scanned layers, according to example embodiments. Referring to FIG. 2, the fMRI system scans the patient's brain using scan cross sections of equal height 220 to produce slices. The slices 220 have the same thickness and volume of the same number voxels 200.

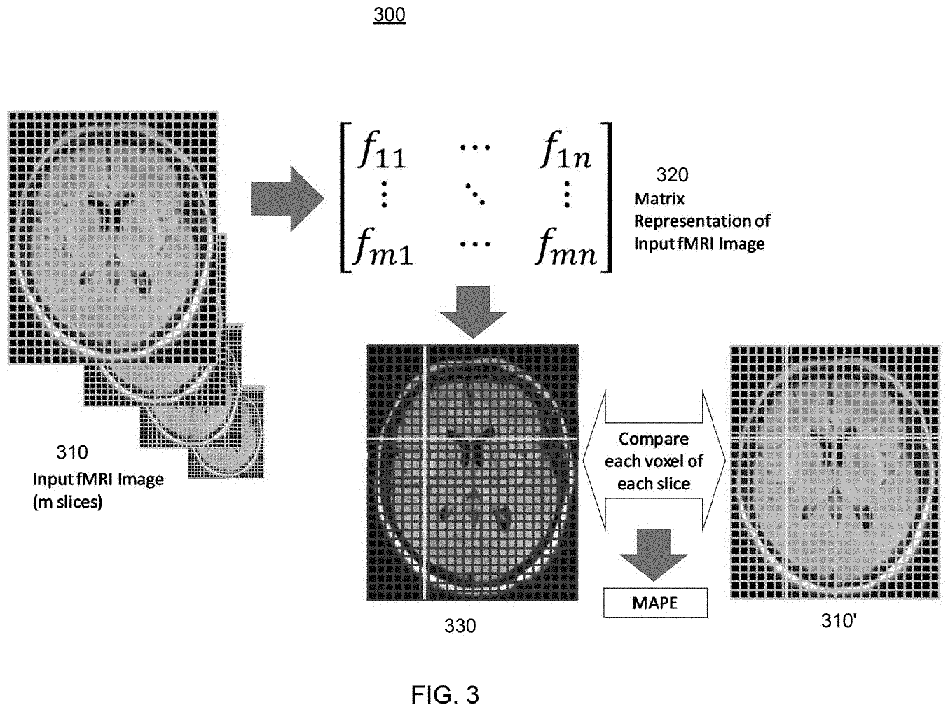

FIG. 3 illustrates fMRI image processing for detection of mental conditions, according to example embodiments. The input fMRI image 310 may consist of m slices. A feature matrix 320 representation of the image 310 is created. The matrix 320 includes a vector representing all features (1 to n) of the same timestep. Each feature of the matrix 320 is indicative of blood oxygen levels of a j^{th} set of voxels of an i^{th} volumetric slice of the fMRI image. The exemplary matrix 320 may include 256 features/voxels recorded over 1024 timesteps. A feature image 320 may be generated based on the matrix 330. Then, the feature image 320 may be compared against each of them slices 310′ voxel by voxel. The correlation level may indicate a mental condition as discussed in more details herein. The error value of comparison may be calculates as a mean absolute percentage error (MAPE), where

$$MAPE = \frac{1}{z} {\sum }_{k = 1}^{z} \vert \frac{{A}_{k} - {F}_{k}}{{A}_{k}} \vert ,$$

where z is a total number of voxels in the set of input fMRI images, Fₖ is the inferred blood oxygen level value of a second voxel k, and Aₖ is the first blood oxygen level value of a first voxel corresponding to the second voxel k. The first predetermined threshold may be set at 9.6%.

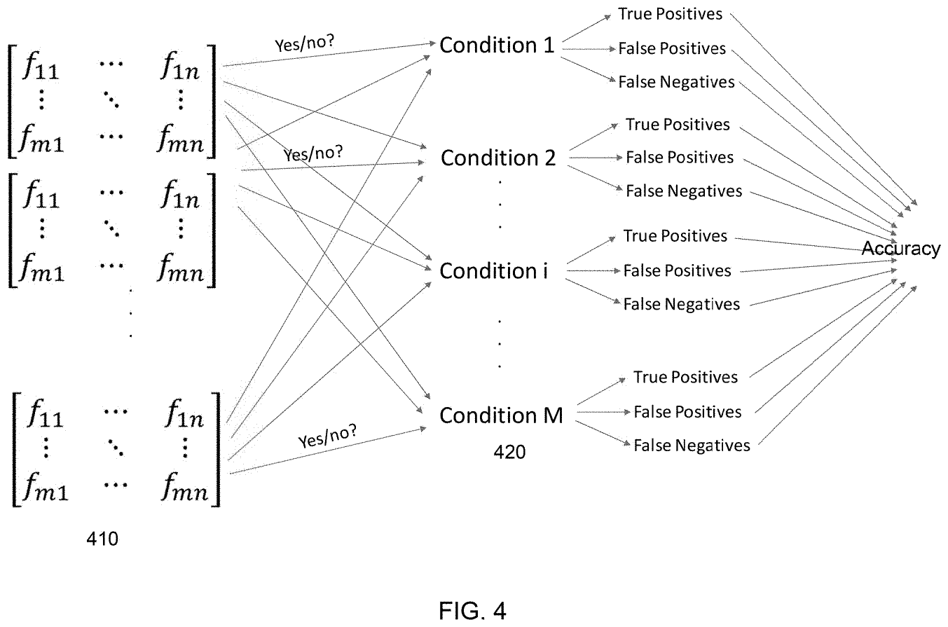

FIG. 4 illustrates a diagram of application of feature/step matrixes for detection of mental conditions, according to example embodiments. As discussed with reference to FIG. 3, the feature/step matrixes 410 may be used for detection of mental conditions 420. Determination of each of the conditions 420 may produce true positive, false positive and false negative values based on accuracy. The accuracy of the determination may be calculated as:

the accuracy value is

$${F}_{1} = \text{?} ,$$

$$\text{?} \text{indicates text missing or illegible when filed}$$

tp is a number of true positive predictions, fp is a number of false positive predictions, and fn is a number of false negative predictions.

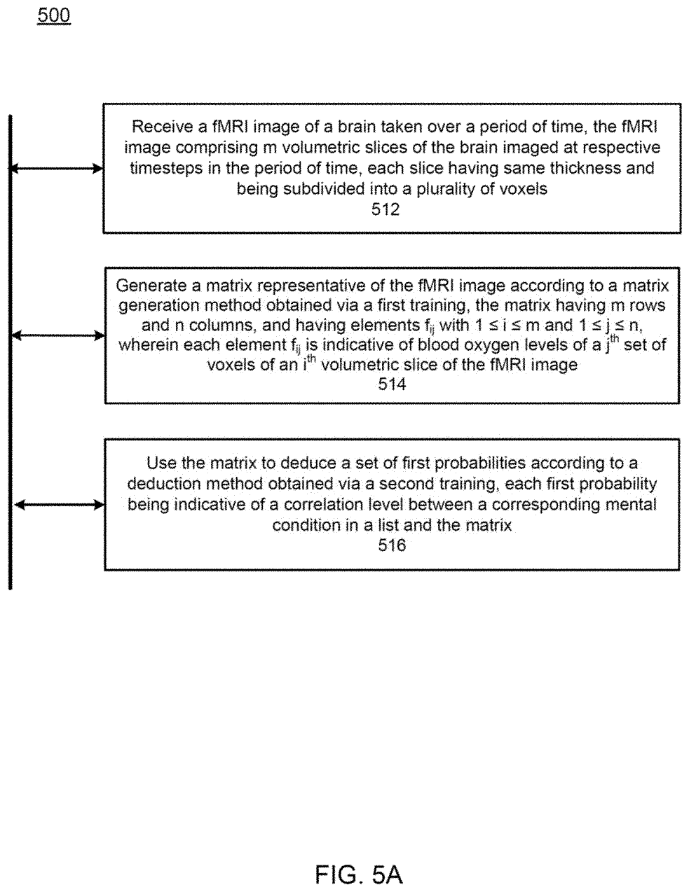

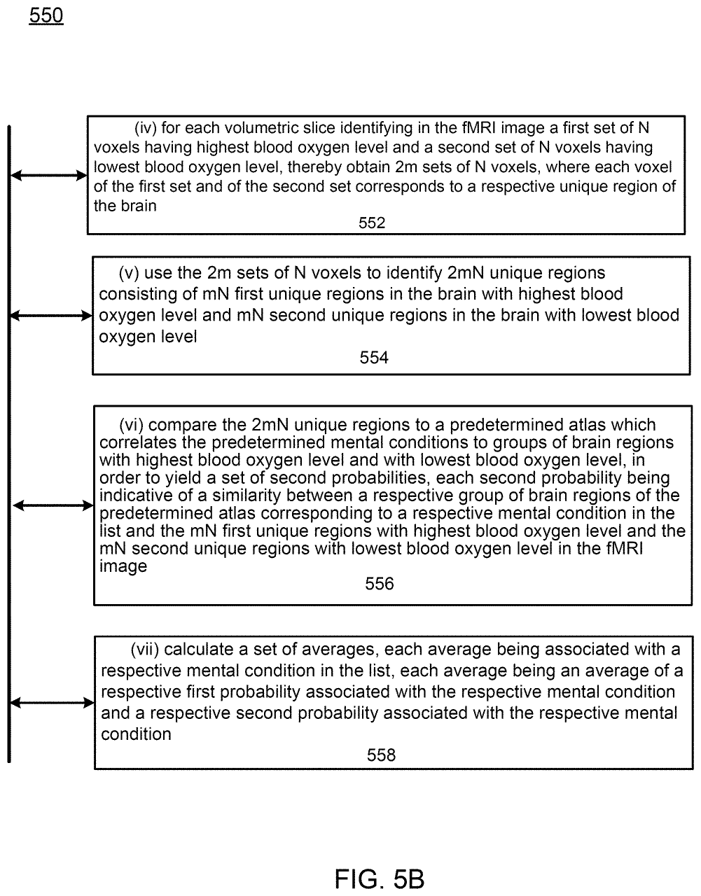

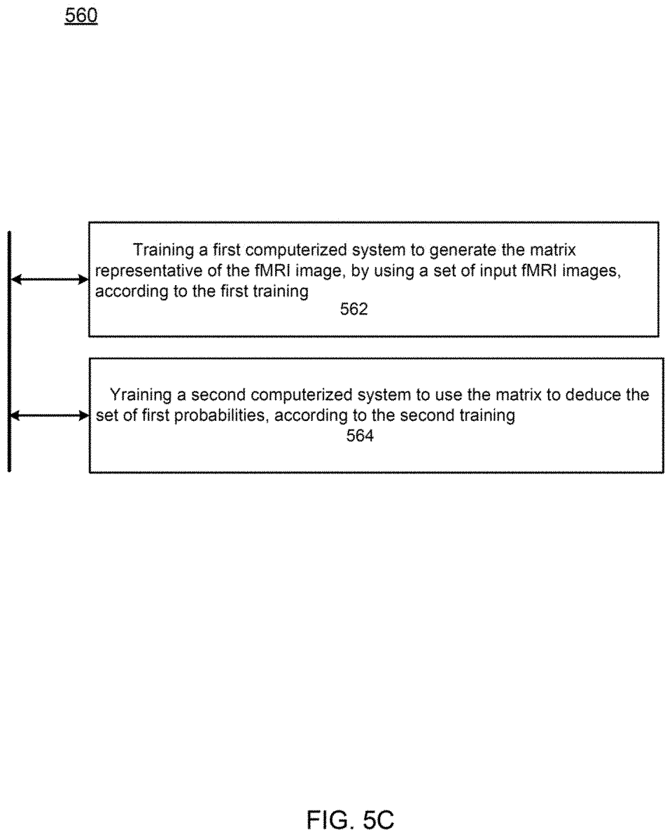

FIG. 5A illustrates a flow diagram 500 of an example method for detection of mental condition based on functional MRI scans, according to example embodiments. Referring to FIG. 5A, the method 500 may include one or more of the steps described below.

FIG. 5A illustrates a flow chart of an example method executed by the diagnostics server 120 (see FIG. 1B). It should be understood that method 500 depicted in FIG. 5A may include additional operations and that some of the operations described therein may be removed and/or modified without departing from the scope of the method 500. The description of the method 500 is also made with reference to the features depicted in FIG. 1B for purposes of illustration. Particularly, the processor 104 of the diagnostics server 120 may execute some or all of the operations included in the method 500.

With reference to FIG. 5A, at block 512, the processor 104 may receive a fMRI image of a brain taken over a period of time, the fMRI image comprising m volumetric slices of the brain imaged at respective timesteps in the period of time, each slice having same thickness and being subdivided into a plurality of voxels. At block 514, the processor 104 may generate a matrix representative of the fMRI image according to a matrix generation method obtained via a first training, the matrix having m rows and n columns, and having elements fᵢⱼ with 1≤i≤m and 1≤j≤n, wherein each element fᵢⱼ is indicative of blood oxygen levels of a j^{th} set of voxels of an i^{th} volumetric slice of the fMRI image. At block 516, the processor 104 may use the matrix to deduce a set of first probabilities according to a deduction method obtained via a second training, each first probability being indicative of a correlation level between a corresponding mental condition in the list and the matrix.

FIG. 5B illustrates a flow diagram 550 of an example method, according to example embodiments. Referring to FIG. 5B, the method 550 may also include one or more of the following steps. At block 552, the processor 104 may for each volumetric slice identifying in the fMRI image a first set of N voxels having highest blood oxygen level and a second set of N voxels having lowest blood oxygen level, obtain 2m sets of N voxels, where each voxel of the first set and of the second set corresponds to a respective unique region of the brain. At block 554, the processor 104 may use the 2m sets of N voxels to identify 2 mN unique regions consisting of mN first unique regions in the brain with highest blood oxygen level and mN second unique regions in the brain with lowest blood oxygen level. At block 556, the processor 104 may compare the 2 mN unique regions to a predetermined atlas which correlates the predetermined mental conditions to groups of brain regions with highest blood oxygen level and with lowest blood oxygen level, in order to yield a set of second probabilities, each second probability being indicative of a similarity between a respective group of brain regions of the predetermined atlas corresponding to a respective mental condition in the list and the mN first unique regions with highest blood oxygen level and the mN second unique regions with lowest blood oxygen level in the fMRI image. At block 558, the processor 104 may calculate a set of averages, each average being associated with a respective mental condition in the list, each average being an average of a respective first probability associated with the respective mental condition and a respective second probability associated with the respective mental condition.

In one exemplary embodiment, the atlas may be a Talairach atlas and N may equal 11. Note that each average may be a geometric average of the respective first probability associated with the respective mental condition and the respective second probability associated with the respective mental condition.

FIG. 5C illustrates a flow diagram 560 of an example method, according to example embodiments. Referring to FIG. 5C, the method 560 may also include one or more of the following steps. At block 562, the processor 104 may train a first computerized system to generate the matrix representative of the fMRI image, by using a set of input fMRI images, according to the first training. At block 562, the processor 104 may train a second computerized system to use the matrix to deduce the set of first probabilities, according to the second training.

According to one embodiment, the training of the first computerized system (i.e., a first model) may execute the following steps:

(a1) providing the set of input fMRI images, each input fMRI image taken over a respective period of time, each input fMRI image comprising respective m first volumetric slices of the brain imaged at respective timesteps in the period of time, each first volumetric slice having same thickness and being subdivided into a plurality of first voxels, and each first voxel having a respective first location and a respective first blood oxygen level value;

(a2) for each of the input fMRI images, instructing the first computerized system to generate a respective matrix having m rows and n columns according to predetermined first guidelines, the matrix having elements fᵢⱼ with 1≤i≤m and 1≤j≤n, wherein each element fᵢⱼ is indicative of blood oxygen levels of a j^{th} set of voxels of an i^{th} volumetric slice of the input fMRI image;

(a3) using each matrix to infer an output representation of the respective input fMRI image, each output representation having m second volumetric slices which correspond to the respective m first volumetric slices, wherein each second volumetric slice is subdivided into a plurality of second voxels corresponding to the plurality of first voxels of the input fMRI image, each second voxel having a respective second location equal to the first location of the corresponding first voxel and a respective inferred blood oxygen level value;

(a4) calculating an error value by comparing each inferred blood oxygen level value of each second voxel to the first blood oxygen level of the corresponding first voxel;

(a5) if the error value is greater than a first predetermined threshold, reporting the error value to the first computerized system and instructing the first computerized system to repeat steps (a2) through (a4) to lower the error value by altering one or more parameters of the first guidelines, until the error value is lower than or equal to the first predetermined threshold;

(a6) if the error value is smaller than or equal to first predetermined threshold, ending training.

Note, that the error value may be calculates as a mean absolute percentage error (MAPE), where

$$MAPE = \frac{1}{z} {\sum }_{k = 1}^{z} \vert \frac{{A}_{k} - {F}_{k}}{{A}_{k}} \vert ,$$

where z is a total number of voxels in the set of input fMRI images, Fₖ is the inferred blood oxygen level value of a second voxel k, and Aₖ is the first blood oxygen level value of a first voxel corresponding to the second voxel k. The first predetermined threshold may be set at 9.6%.

The training of the second computerized system (i.e., a second model) may execute the following steps:

(b1) after the first training is complete, receiving at least a subset of the set of matrices corresponding to input fMRI images of brains of patients that are known to have one or more of the predetermined mental conditions;

(b2) instructing the second computerized system to use the matrices in the subset to predict whether each matrix corresponds to any of the one or more of the predetermined mental conditions, via second guidelines;

(b3) calculating an accuracy value for the subset, by comparing predictions generated at (b2) to the known conditions corresponding to each matrix in the subset;

(b4) if the accuracy value is lower than a second predetermined threshold, reporting the accuracy value to the second computerized system and instructing the second computerized system to repeat steps (b2) and (b3) to increase the accuracy value by altering one or more parameters of the second guidelines, until the accuracy value is greater than or equal to the second predetermined threshold;

(b5) if the accuracy value is greater than or equal to second predetermined threshold, ending training.

Note, that the accuracy value may be calculated as

$${F}_{1} = \text{?} ,$$

$$\text{?} \text{indicates text missing or illegible when filed}$$

tp is a number oft predictions, fp is a number of false positive predictions, and fn is a number of false negative predictions. The second predetermined threshold may be set at 90%.

According to one embodiment, during the training of the second AI model to predict conditions from matrices, a set of 1024×256 matrices is generated, in which each matrix is representative of a respective patient's known mental condition and another set of matrices to test the generalization capability to unknown mental conditions.

The above embodiments may be implemented in hardware, in a computer program executed by a processor, in firmware, or in a combination of the above. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

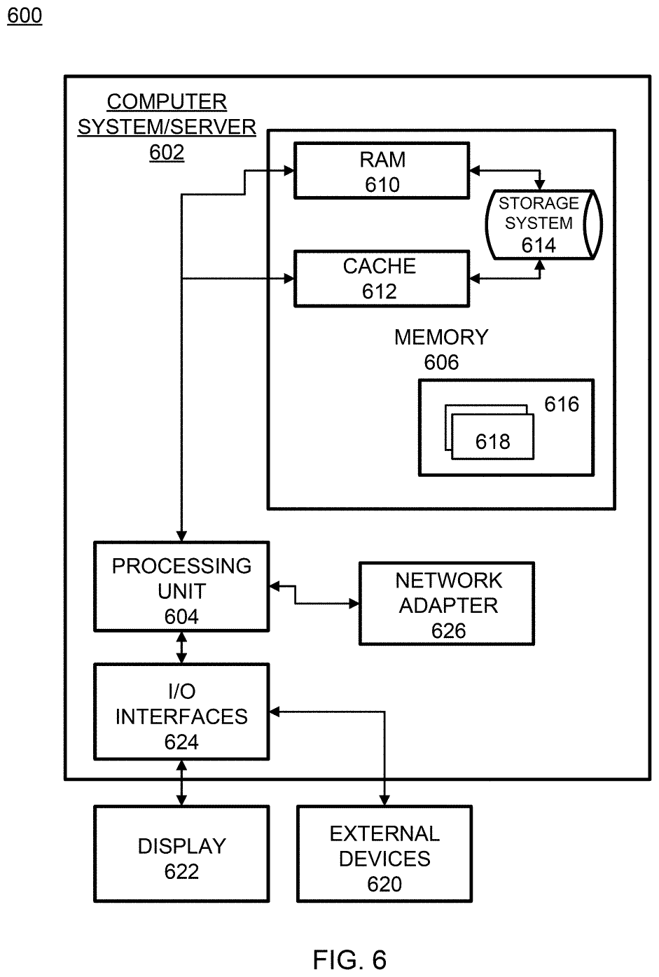

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 6 illustrates an example computer system/server node 600, which may represent or be integrated in any of the above-described components, etc.

FIG. 6 is not intended to suggest any limitation as to the scope of use or functionality of embodiments of the application described herein. Regardless, the computing node 600 is capable of being implemented and/or performing any of the functionality set forth hereinabove.

In the computing node 600 there is a computer system/server 602, which is operational with numerous other general purposes or special purpose computing system environments or configurations. Examples of well-known computing systems, environments, and/or configurations that may be suitable for use with computer system/server 602 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 602 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 602 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, the computer system/server 602 may be used in cloud computing node 600 shown in the form of a general-purpose computing device. The components of the computer system/server 602 may include, but are not limited to, one or more processors or processing units 604, a system memory 606, and a bus that couples various system components including system memory 606 to processor 604.

The bus represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

The exemplary computer system/server 602 typically includes a variety of computer system readable media. Such media may be any available media that is accessible by the computer system/server 602, and it includes both volatile and non-volatile media, removable and non-removable media. System memory 606, in one embodiment, implements the flow diagrams of the other figures. The system memory 606 can include computer system readable media in the form of volatile memory, such as random-access memory (RAM) 610 and/or cache memory 612. The computer system/server 602 may further include other removable/non-removable, volatile/non-volatile computer system storage media. By way of example only, storage system 614 can be provided for reading from and writing to a non-removable, non-volatile magnetic media (not shown and typically called a “hard drive”). Although not shown, a magnetic disk drive for reading from and writing to a removable, non-volatile magnetic disk, and an optical disk drive for reading from or writing to a removable, non-volatile optical disk such as a CD-ROM, DVD-ROM or other optical media can be provided. In such instances, each can be connected to the bus by one or more data media interfaces. As will be further depicted and described below, memory 606 may include at least one program product having a set (e.g., at least one) of program modules that are configured to carry out the functions of various embodiments of the application.

Program/utility 616, having a set (at least one) of program modules 618, may be stored in memory 606 by way of example, and not limitation, as well as an operating system, one or more application programs, other program modules, and program data. Each of the operating system, one or more application programs, other program modules, and program data or some combination thereof, may include an implementation of a networking environment. Program modules 618 generally carry out the functions and/or methodologies of various embodiments of the application as described herein.

As will be appreciated by one skilled in the art, aspects of the present application may be embodied as a system, method, or computer program product. Accordingly, aspects of the present application may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present application may take the form of a computer program product embodied in one or more computer readable medium(s) having computer readable program code embodied thereon.

The computer system/server 602 may also communicate with one or more external devices 620 such as a keyboard, a pointing device, a display 622, etc.; one or more devices that enable a user to interact with computer system/server 602; and/or any devices (e.g., network card, modem, etc.) that enable computer system/server 602 to communicate with one or more other computing devices. Such communication can occur via I/O interfaces 624. Still yet, the computer system/server 602 can communicate with one or more networks such as a local area network (LAN), a general wide area network (WAN), and/or a public network (e.g., the Internet) via network adapter 626. As depicted, network adapter 626 communicates with the other components of computer system/server 602 via a bus. It should be understood that although not shown, other hardware and/or software components could be used in conjunction with computer system/server 602. Examples include, but are not limited to: microcode, device drivers, redundant processing units, external disk drive arrays, RAID systems, tape drives, and data archival storage systems, etc.

Although an exemplary embodiment of at least one of a system, method, and non-transitory computer readable medium has been illustrated in the accompanied drawings and described in the foregoing detailed description, it will be understood that the application is not limited to the embodiments disclosed, but is capable of numerous rearrangements, modifications, and substitutions as set forth and defined by the following claims. For example, the capabilities of the system of the various figures can be performed by one or more of the modules or components described herein or in a distributed architecture and may include a transmitter, recipient or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a Smart phone or any other suitable computing device, or combination of devices. Presenting the above-described functions as being performed by a “system” is not intended to limit the scope of the present application in any way but is intended to provide one example of many embodiments. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large-scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed but is merely representative of selected embodiments of the application.

One having ordinary skill in the art will readily understand that the above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms, etc.) thereto.

1. A system, comprising:
a processor of a diagnostics server node connected to an image processor of a functional MRI (NMI) system and to a first computerized system and to a second computerized system;
a memory on which are stored machine readable instructions that when executed by the processor, cause the processor to:
(i) provide a fMRI image of a brain taken over a period of time, the fMRI image comprising m volumetric slices of the brain imaged at respective time steps in the period of time, each slice having same thickness and being subdivided into a plurality of voxels;
(ii) generate a matrix representative of the fMRI image according to a matrix generation method obtained via a first training, the matrix having m rows and n columns, and having elements fᵢⱼ with 1≤i≤m and 1≤j≤n, wherein each element fᵢⱼ is indicative of blood oxygen levels of a i^{th} set of voxels of an i^{th} volumetric slice of the fMRI image; and
(iii) use the matrix to obtain a set of first probabilities according to a deduction method obtained via a second training, each first probability being indicative of a correlation level between a corresponding mental condition in a list and the matrix.

2. The system of claim 1, wherein the instructions further cause the processor to:
(iv) for each volumetric slice identify in the fMRI image a first set of N voxels having highest blood oxygen level and a second set of N voxels having lowest blood oxygen level, thereby obtain 2m sets of N voxels, where each voxel of the first set and of the second set corresponds to a respective unique region of the brain;
(v) use the 2m sets of N voxels to identify 2 mN unique regions consisting of mN first unique regions in the brain with highest blood oxygen level and mN second unique regions in the brain with lowest blood oxygen level;
(vi) compare the 2 mN unique regions to a predetermined atlas which correlates the predetermined mental conditions to groups of brain regions with highest blood oxygen level and with lowest blood oxygen level, in order to yield a set of second probabilities, each second probability being indicative of a similarity between a respective group of brain regions of the predetermined atlas corresponding to a respective mental condition in the list and the mN first unique regions with highest blood oxygen level and the mN second unique regions with lowest blood oxygen level in the fMRI image; and
(vii) calculate a set of averages, each average being associated with a respective mental condition in the list, each average being an average of a respective first probability associated with the respective mental condition and a respective second probability associated with the respective mental condition.

3. The system of claim 2, wherein the atlas is a Talairach atlas.

4. The system of claim 1, wherein each average is a geometric average of the respective first probability associated with the respective mental condition and the respective second probability associated with the respective mental condition.

5. The system of claim 1, wherein the instructions further cause the processor to, prior to step (i):
(a) train the first computerized system to generate the matrix representative of the fMRI image, by using a set of input fMRI images, according to the first training; and
(b) train the second computerized system to use the matrix to obtain the set of first probabilities, according to the second training.

6. The system of claim 5, wherein the first training comprises:
(a1) providing the set of input fMRI images, each input fMRI image taken over a respective period of time, each input fMRI image comprising respective m first volumetric slices of the brain imaged at respective timesteps in the period of time, each first volumetric slice having same thickness and being subdivided into a plurality of first voxels, and each first voxel having a respective first location and a respective first blood oxygen level value;
(a2) for each of the input fMRI images, instructing the first computerized system to generate a respective matrix having m rows and n columns according to predetermined first guidelines, the matrix having elements fᵢⱼ with 1≤i≤m and 1≤j≤n, wherein each element fᵢⱼ is indicative of blood oxygen levels of a j^{th} set of voxels of an i^{th} volumetric slice of the input fMRI image;
(a3) using each matrix to infer an output representation of the respective input fMRI image, each output representation having m second volumetric slices which correspond to the respective m first volumetric slices, wherein each second volumetric slice is subdivided into a plurality of second voxels corresponding to the plurality of first voxels of the input fMRI image, each second voxel having a respective second location equal to the first location of the corresponding first voxel and a respective inferred blood oxygen level value;
(a4) calculating an error value by comparing each inferred blood oxygen level value of each second voxel to the first blood oxygen level of the corresponding first voxel;
(a5) if the error value is greater than a first predetermined threshold, reporting the error value to the first computerized system and instructing the first computerized system to repeat steps (a2) through (a4) to lower the error value by altering one or more parameters of the first guidelines, until the error value is lower than or equal to the first predetermined threshold; and
(a6) if the error value is smaller than or equal to first predetermined threshold, ending training.

7. The system of claim 6, wherein the error value is a mean absolute percentage error (MAPE), where
$$MAPE = \frac{1}{z} {\sum }_{k = 1}^{z} \vert \frac{{A}_{k} - {F}_{k}}{{A}_{k}} \vert ,$$
where z is a total number of voxels in the set of input fMRI images, Fₖ is the inferred blood oxygen level value of a second voxel k, and Aₖ is the first blood oxygen level value of a first voxel corresponding to the second voxel k.

8. The system of claim 5, wherein the second training comprises:
(b1) after the first training is complete, receiving at least a subset of the set of matrices corresponding to input fMRI images of brains of patients that are known to have one or more of the predetermined mental conditions;
(b2) instructing the second computerized system to use the matrices in the subset to predict whether each matrix corresponds to any of the one or more of the predetermined mental conditions, via second guidelines;
(b3) calculating an accuracy value for the subset, by comparing predictions generated at (b2) to the known conditions corresponding to each matrix in the subset;
(b4) if the accuracy value is lower than a second predetermined threshold, reporting the accuracy value to the second computerized system and instructing the second computerized system to repeat steps (b2) and (b3) to increase the accuracy value by altering one or more parameters of the second guidelines, until the accuracy value is greater than or equal to the second predetermined threshold; and
(b5) if the accuracy value is greater than or equal to second predetermined threshold, ending training.

9. The system of claim 8, wherein the accuracy value is
$${F}_{1} = \text{?} ,$$
$$\text{?} \text{indicates text missing or illegible when filed}$$
tp is a number of correct predictions, fp is a number of false positive predictions, and fn is a number of false negative predictions.

10. A method for detection of mental condition based on functional MRI scans, the method comprising:
(i) receiving, by a diagnostics server, a fMRI image of a brain taken over a period of time, the fMRI image comprising m volumetric slices of the brain imaged at respective time steps in the period of time, each slice having same thickness and being subdivided into a plurality of voxels;
(ii) generating, by the diagnostics server, a matrix representative of the fMRI image according to a matrix generation method obtained via a first training, the matrix having m rows and n columns, and having elements fᵢⱼ with 1≤i≤m and 1≤j≤n, wherein each element fᵢⱼ is indicative of blood oxygen levels of a j^{th} set of voxels of an i^{th} volumetric slice of the fMRI image; and
(iii) using, by the diagnostics server, the matrix to obtain a set of first probabilities according to a deduction method obtained via a second training, each first probability being indicative of a correlation level between a corresponding mental condition in a list and the matrix.

11. The method of claim 10, further comprising:
(iv) for each volumetric slice identifying in the fMRI image a first set of N voxels having highest blood oxygen level and a second set of N voxels having lowest blood oxygen level, thereby obtaining 2m sets of N voxels, where each voxel of the first set and of the second set corresponds to a respective unique region of the brain;
(v) using the 2m sets of N voxels to identify 2 mN unique regions consisting of mN first unique regions in the brain with highest blood oxygen level and mN second unique regions in the brain with lowest blood oxygen level;
(vi) comparing the 2 mN unique regions to a predetermined atlas which correlates the predetermined mental conditions to groups of brain regions with highest blood oxygen level and with lowest blood oxygen level, in order to yield a set of second probabilities, each second probability being indicative of a similarity between a respective group of brain regions of the predetermined atlas corresponding to a respective mental condition in the list and the mN first unique regions with highest blood oxygen level and the mN second unique regions with lowest blood oxygen level in the fMRI image; and
(vii) calculating a set of averages, each average being associated with a respective mental condition in the list, each average being an average of a respective first probability associated with the respective mental condition and a respective second probability associated with the respective mental condition.

12. The method of claim 10, further comprising, prior to step (i):
(a) training a first computerized system to generate the matrix representative of the fMRI image, by using a set of input fMRI images, according to the first training; and
(b) training a second computerized system to use the matrix to obtain the set of first probabilities, according to the second training.

13. The method of claim 12, wherein the first training comprises:
(a1) providing the set of input fMRI images, each input fMRI image taken over a respective period of time, each input fMRI image comprising respective m first volumetric slices of the brain imaged at respective timesteps in the period of time, each first volumetric slice having same thickness and being subdivided into a plurality of first voxels, and each first voxel having a respective first location and a respective first blood oxygen level value;
(a2) for each of the input fMRI images, instruct the first computerized system to generating a respective matrix having m rows and n columns according to predetermined first guidelines, the matrix having elements fᵢⱼ with 1≤i≤m and 1≤j≤n, wherein each element fᵢⱼ is indicative of blood oxygen levels of a j^{th} set of voxels of an i^{th} volumetric slice of the input fMRI image;
(a3) using each matrix to infer an output representation of the respective input fMRI image, each output representation having m second volumetric slices which correspond to the respective m first volumetric slices, wherein each second volumetric slice is subdivided into a plurality of second voxels corresponding to the plurality of first voxels of the input fMRI image, each second voxel having a respective second location equal to the first location of the corresponding first voxel and a respective inferred blood oxygen level value;
(a4) calculating an error value by comparing each inferred blood oxygen level value of each second voxel to the first blood oxygen level of the corresponding first voxel;
(a5) if the error value is greater than a first predetermined threshold, reporting the error value to the first computerized system and instructing the first computerized system to repeat steps (a2) through (a4) to lower the error value by altering one or more parameters of the first guidelines, until the error value is lower than or equal to the first predetermined threshold; and
(a6) if the error value is smaller than or equal to first predetermined threshold, ending training.

14. The method of claim 12, wherein the second training comprises:
(b1) after the first training is complete, receiving at least a subset of the set of matrices corresponding to input fMRI images of brains of patients that are known to have one or more of the predetermined mental conditions;
(b2) instructing the second computerized system to use the matrices in the subset to predict whether each matrix corresponds to any of the one or more of the predetermined mental conditions, via second guidelines;
(b3) calculating an accuracy value for the subset, by comparing predictions generated at (b2) to the known conditions corresponding to each matrix in the subset;
(b4) if the accuracy value is lower than a second predetermined threshold, reporting computerized system to repeat steps (b2) and (b3) to increase the accuracy value by altering one or more parameters of the second guidelines, until the accuracy value is greater than or equal to the second predetermined threshold; and
(b5) if the accuracy value is greater than or equal to second predetermined threshold, ending training.

15. A non-transitory computer readable medium comprising instructions, that when read by a processor, cause the processor to perform:
(i) providing a fMRI image of a brain taken over a period of time, the fMRI image comprising m volumetric slices of the brain imaged at respective time steps in the period of time, each slice having same thickness and being subdivided into a plurality of voxels;
(ii) generating a matrix representative of the fMRI image according to a matrix generation method obtained via a first training, the matrix having m rows and n columns, and having elements fᵢⱼ with 1≤i≤m and 1≤j≤n, wherein each element fᵢⱼ is indicative of blood oxygen levels of a i^{th} set of voxels of an i^{th} volumetric slice of the fMRI image; and
(iii) using the matrix to obtain a set of first probabilities according to a deduction method obtained via a second training, each first probability being indicative of a correlation level between a corresponding mental condition in a list and the matrix.

16. The non-transitory computer readable medium of claim 15, further comprising instructions, that when read by the processor, cause the processor to:
(iv) for each volumetric slice identify in the fMRI image a first set of N voxels having highest blood oxygen level and a second set of N voxels having lowest blood oxygen level, thereby obtain 2m sets of N voxels, where each voxel of the first set and of the second set corresponds to a respective unique region of the brain;
(v) use the 2m sets of N voxels to identify 2 mN unique regions consisting of mN first unique regions in the brain with highest blood oxygen level and mN second unique regions in the brain with lowest blood oxygen level;
(vi) compare the 2 mN unique regions to a predetermined atlas which correlates the predetermined mental conditions to groups of brain regions with highest blood oxygen level and with lowest blood oxygen level, in order to yield a set of second probabilities, each second probability being indicative of a similarity between a respective group of brain regions of the predetermined atlas corresponding to a respective mental condition in the list and the mN first unique regions with highest blood oxygen level and the mN second unique regions with lowest blood oxygen level in the fMRI image; and
(vii) calculate a set of averages, each average being associated with a respective mental condition in the list, each average being an average of a respective first probability associated with the respective mental condition and a respective second probability associated with the respective mental condition.

17. The non-transitory computer readable medium of claim 15, further comprising instructions, that when read by the processor, cause the processor to:
(a) train a first model to generate the matrix representative of the fMRI image, by using a set of input fMRI images, according to the first training; and
(b) train a second model to use the matrix to obtain the set of first probabilities, according to the second training.

18. The non-transitory computer readable medium of claim 17, wherein training of the first model comprises:
(a1) providing the set of input fMRI images, each input fMRI image taken over a respective period of time, each input fMRI image comprising respective m first volumetric slices of the brain imaged at respective timesteps in the period of time, each first volumetric slice having same thickness and being subdivided into a plurality of first voxels, and each first voxel having a respective first location and a respective first blood oxygen level value;
(a2) for each of the input fMRI images, instruct the first computerized system to generating a respective matrix having m rows and n columns according to predetermined first guidelines, the matrix having elements fᵢⱼ with 1≤i≤m and 1≤j≤n, wherein each element fᵢⱼ is indicative of blood oxygen levels of a j^{th} set of voxels of an i^{th} volumetric slice of the input fMRI image;
(a3) using each matrix to infer an output representation of the respective input fMRI image, each output representation having m second volumetric slices which correspond to the respective m first volumetric slices, wherein each second volumetric slice is subdivided into a plurality of second voxels corresponding to the plurality of first voxels of the input fMRI image, each second voxel having a respective second location equal to the first location of the corresponding first voxel and a respective inferred blood oxygen level value;
(a4) calculating an error value by comparing each inferred blood oxygen level value of each second voxel to the first blood oxygen level of the corresponding first voxel;
(a5) if the error value is greater than a first predetermined threshold, reporting the error value to the first computerized system and instructing the first computerized system to repeat steps (a2) through (a4) to lower the error value by altering one or more parameters of the first guidelines, until the error value is lower than or equal to the first predetermined threshold; and
(a6) if the error value is smaller than or equal to first predetermined threshold, ending the training of the first model.

19. The non-transitory computer readable medium of claim 18, further comprising instructions, that when read by the processor, cause the processor to calculate the error value as a mean absolute percentage error (MAPE), where
$$MAPE = \frac{1}{z} {\sum }_{k = 1}^{z} \vert \frac{{A}_{k} - {F}_{k}}{{A}_{k}} \vert ,$$
where z is a total number of voxels in the set of input fMRI images, Fₖ is the inferred blood oxygen level value of a second voxel k, and Aₖ is the first blood oxygen level value of a first voxel corresponding to the second voxel k.

20. The non-transitory computer readable medium of claim 17, wherein training of the second model comprises:
(b1) after the training of the first model is complete, receiving at least a subset of the set of matrices corresponding to input fMRI images of brains of patients that are known to have one or more of the predetermined mental conditions;
(b2) instructing the second model to use the matrices in the subset to predict whether each matrix corresponds to any of the one or more of the predetermined mental conditions, via second guidelines;
(b3) calculating an accuracy value for the subset, by comparing predictions generated at (b2) to the known conditions corresponding to each matrix in the subset;
(b4) if the accuracy value is lower than a second predetermined threshold, repeating steps (b2) and (b3) to increase the accuracy value by altering one or more parameters of the second guidelines, until the accuracy value is greater than or equal to the second predetermined threshold; and
(b5) if the accuracy value is greater than or equal to second predetermined threshold, ending the training of the second model,
wherein the accuracy value is
$${F}_{1} = \text{?} ,$$
$$\text{?} \text{indicates text missing or illegible when filed}$$
tp is a number of correct predictions, fp is a number of false positive predictions, and fn is a number of false negative predictions.
